(19) United States
(12) Patent Application Publication
(10) Pub. No.: US 2014/0350111 A1
(43) Pub. Date: Nov. 27, 2014

(54) METHODS FOR TREATMENT AND PROPHYLAXIS AGAINST HEMORRHOIDS

(71) Applicant: Guy Alan Hale, Danville, CA (US)

(72) Inventor: Guy Alan Hale, Danville, CA (US)

(21) Appl. No.: 14/343,806

(22) PCT Filed: Sep. 7, 2012
(86) PCT No.: PCT/US2012/054275

§ 371 Date: Aug. 5, 2014

Related U.S. Application Data
(60) Provisional application No. 61/531,699, filed on Sep. 7, 2011.

(51) Int. Cl. A61K 31/192

(52) U.S. Cl. CPC A61K 31/192
USPC 514/569

(57) ABSTRACT

Methods for treatment of and prophylaxis against hemorrhoids are disclosed, including methods of treatment of and prophylaxis against hemorrhoids comprising administering naproxen to an individual.

### RELATED APPLICATION

The present application claims priority to U.S. Provisional Application No. 61/531,699, filed on Sep. 7, 2011 and titled “Methods for Treatment of and Prophylaxis Against Hemorrhoids,” the entirety of which is hereby incorporated by reference.

### TECHNICAL FIELD

The present disclosure is directed to methods for treatment of and prophylaxis against hemorrhoids.

### BACKGROUND

Hemorrhoids are very common, especially during pregnancy and after childbirth. They result from increased pressure in the veins of the anus. The pressure causes the veins to swell, making them painful, particularly when an affected individual is sitting.

Hemorrhoids may be caused by straining during bowel movements, constipation, sitting for long periods of time, anal infections, and certain diseases, such as liver cirrhosis.

Hemorrhoids may be inside or outside the body. Internal hemorrhoids occur just inside the anus, at the beginning of the rectum. External hemorrhoids occur at the anal opening and may hang outside the anus.

Symptoms of hemorrhoids include anal itching, anal ache or pain, especially while sitting, bright red blood or pus on toilet tissue, stool, or in the toilet bowl, pain during bowel movements, a foul odor, and one or more hard tender lumps near the anus.

There are no known cures or methods of prophylaxis against hemorrhoids. An investigation of the available data reveals that there are only products that help reduce the pain and swelling of hemorrhoids. Current treatments for hemorrhoids include over-the-counter corticosteroid creams to help reduce pain and swelling, hemorrhoid creams with lidocaine to help reduce pain, stool softeners to help reduce straining and constipation, witch hazel (applied with cotton swabs) to reduce itching, and sitz baths.

It is estimated that there are over 500 million people in the world who suffer from hemorrhoids. Therefore, it is readily apparent that there is a great need for effective hemorrhoid treatments.

### SUMMARY

In one aspect, the present disclosure is related to methods of treating an individual suffering from hemorrhoids. In one embodiment, the method comprises administering to the individual a therapeutically effective amount of a nonsteroidal anti-inflammatory drug (“NSAID”), naproxen, wherein administering the naproxen to the individual leads to abatement of a hemorrhoid symptom in the individual.

In certain embodiments, naproxen is administered to the individual in a dose of from about 1 mg/kg/day to about 40 mg/kg/day. In other embodiments, naproxen is administered to the individual in a dose of from about 4 mg/kg/day to about 10 mg/kg/day.

In certain embodiments, the naproxen is administered to the individual at approximately twelve-hour intervals. In other embodiments, the naproxen is administered to the individual at approximately six- to eight-hour intervals. In another embodiment, the naproxen is administered to the individual once, twice or three times per day.

In certain embodiments, the method comprises administering from about 220 mg naproxen to about 550 mg naproxen twice per day. For example, 220, 250, 275, 375, 440, 500, or 550 mg naproxen sodium may be administered to the individual twice per day, at approximately twelve hour intervals. In some embodiment, 440 mg naproxen sodium is administered to the individual twice per day, at approximately twelve-hour intervals. In other embodiments, the method comprises administering 440 mg naproxen sodium to the individual three times per day, at approximately six to eight hour intervals.

In certain embodiments, the naproxen is a controlled-release or extended-release form administered once or twice daily. For example, from about 375 mg to about 1000 mg of a controlled-release form of naproxen may be administered once daily.

In a second aspect, this disclosure is related to methods of prophylaxis against hemorrhoids. In one embodiment, this disclosure provides a method of prophylaxis against hemorrhoids in an individual at risk of developing hemorrhoids, comprising administering to the individual a therapeutically effective amount of naproxen, wherein administering the naproxen to the individual is prophylactic against the development of a hemorrhoid symptom in the individual.

In certain embodiments, naproxen is administered to the individual in a dose of from about 0.25 mg/kg/day to about 20 mg/kg/day. In other embodiments, naproxen is administered to the individual in a dose of from about 0.5 mg/kg/day to about 5 mg/kg/day.

In certain embodiments, the naproxen is administered to the individual every day. In one such embodiment, 220 mg naproxen is administered to the individual every day.

In certain embodiments, the naproxen is administered to the individual at a three-day interval. For example, naproxen may be administered in a dose of from about 0.5 mg/kg to about 5 mg/kg at a three-day interval. In other embodiments, 440 mg naproxen is administered to the individual at a three-day interval.

### DETAILED DESCRIPTION OF PREFERRED EMBODIMENTS

This disclosure is related to methods for treatment of and prophylaxis against hemorrhoids. It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

Definitions

“Nonsteroidal anti-inflammatory drug” or “NSAID” is defined as a non-steroidal drug that inhibits the production of prostaglandins by cyclooxygenase enzymes. NSAIDS are a well-known and well-characterized class of drugs. The pharmacokinetic profiles, and toxicity profiles of these drugs are known in the art. Commonly used NSAIDS include aspirin, ibuprofen, and naproxen.

“Naproxen” is defined as the chemical identified by CAS registry number 22204-53-1, or any salt, solvate, hydrate or any other bioavailable form of the compound known in the art. One form of naproxen in common use is the sodium salt of naproxen, or naproxen sodium.

A “controlled-release form of naproxen” is defined to include pharmaceutical formulations of naproxen and drug delivery technology for naproxen that affect the absorption, half-life, physiological levels, targeting, and/or elimination of naproxen. A controlled-release form of naproxen may, for example, speed the absorption of the naproxen. A controlled-release form of naproxen may also be used to decrease the frequency of dosing. Controlled-release forms of naproxen sodium, such as Naprelan®, are known in the art.

“Abatement of a hemorrhoid symptom in an individual” means a reduction in the severity of any hemorrhoid symptom known in the art. The reduction may be a subjective reduction (meaning the symptoms are reduced as perceived by the individual) or the reduction may be measured by more quantitative means (for example, by a measured reduction in the swelling of veins).

An individual is “at risk of developing hemorrhoids” when that individual presents with any condition known to be associated with hemorrhoids (for example, constipation, pregnancy, or diseases such as anal infections or liver cirrhosis), or when the individual has previously suffered from hemorrhoids, or when the individual engages in any activities known to be associated with the development of hemorrhoids (for example, straining during bowel movements or sitting for long periods of time).

A composition provides “prophylaxis against” or is “prophylactic against” a condition or symptom if when the composition is administered in a therapeutically effective amount to an individual, the probability of that individual developing the condition or symptom is reduced compared to individuals who were not treated with the composition.

When used in describing a dosing regimen, an “interval” represents a time that is elapsed between one or more successive doses, with no intervening doses. For example, administering naproxen to an individual at a six-hour interval means that naproxen is given to the individual at a given time, and then again approximately six hours from the time of the first dose, without an intervening dose. Likewise, for example, administering naproxen to an individual at a three-day interval means that naproxen is administered to an individual at a given time, and then again three days after the first dose, without intervening doses.

Methods

A first aspect of the disclosure relates to methods of treating hemorrhoids.

In certain embodiments, this disclosure provides for a method of treating an individual suffering from hemorrhoids comprising administering to the individual a therapeutically effective amount of naproxen. The naproxen may be administered in one or more of several ways known in the art, including orally, for example, in the form of one or more pills or tablets, or in drop, elixir, suspension, or other liquid form; topically, for example, in the form of a cream or lotion applied to the anus; or rectally, for example, in the form of a suppository inserted into the rectum. Other methods of administration known in the art, for example injection, inhalation, or sublingual administration, may be used.

The pharmacokinetic profiles and toxicity profiles of naproxen are well known in the art. Therefore, in light of the teachings of the present disclosure, a skilled person would be able to determine the ranges of therapeutic amounts of naproxen that would be effective for treating hemorrhoids. For example, in light of the teachings of the present disclosure, a skilled person may administer naproxen to individuals suffering from hemorrhoids in varying doses to determine which doses are effective for treatment of hemorrhoids.

Controlled-release forms of naproxen are also known in the art. In certain embodiments, from about 375 mg to about 1000 mg of a controlled-release form of naproxen is administered once daily. In other embodiments, from about 750 mg to about 1000 mg of a controlled-release form of naproxen is administered once daily.

In certain embodiments, the method comprises administering to an individual naproxen in a dose of from about 1 mg/kg/day to about 40 mg/kg/day. In other embodiments, the method comprises administering to the individual naproxen in a dose of from about 4 mg/kg/day to about 10 mg/kg/day. In some embodiments, the method comprises administering to the individual naproxen in an amount between about 100 mg and 2000 mg per day. In other embodiments, the method comprises administering to the individual naproxen in an amount between about 220 mg and 1760 mg per day, between about 440 mg and 1320 mg per day, or between about 880 mg and 1320 mg per day.

In certain embodiments, the method comprises administering naproxen to the individual in a dose of from about 220 mg to about 1320 mg. In one embodiment, the method comprises administering naproxen to the individual in a 220 mg dose. In another embodiment, the method comprises administering naproxen to the individual in a 440 mg dose. In yet another embodiment, the method comprises administering naproxen to the individual in a 660 mg dose.

In some embodiments, the naproxen is administered daily. In other embodiments, naproxen is administered more than once a day. In some embodiments, naproxen is administered twice a day. In some embodiments, naproxen is administered three times a day. In one embodiment, the method comprises administering naproxen to the individual at twelve-hour intervals. In other embodiments, the method comprises administering naproxen to the individual at six-hour intervals.

In certain embodiment, administering naproxen to the individual leads to abatement of a hemorrhoid symptom in the individual. In some embodiments, administering naproxen to the individual leads to abatement of a hemorrhoid symptom in the individual that is not pain or inflammation. In some embodiments, administering naproxen to the individual leads to abatement of anal itching. In other embodiments, administering naproxen to the individual leads to abatement of bleeding from the rectal area. In other embodiments, administering naproxen to the individual leads to abatement of pus discharge from the rectal area. In other embodiments, administering naproxen to the individual leads to abatement of a foul odor associated with hemorrhoids. In other embodiments, administering naproxen to the individual leads to abatement of lumps near the anus.

In a further aspect, this disclosure relates to methods of prophylaxis against hemorrhoids.

In one embodiment, this disclosure provides for a method of prophylaxis against hemorrhoids in an individual at risk of developing hemorrhoids, comprising administering to the individual a therapeutically effective amount of naproxen.

The pharmacokinetic profiles and toxicity profiles of naproxen are known in the art. Therefore, in light of the teachings and examples provided by the present disclosure, a skilled person would be able to determine the ranges of therapeutic amounts of naproxen that would be effective for prophylaxis against hemorrhoids. For example, in light of the teachings of the present disclosure, a skilled person may administer naproxen to individuals at risk of developing hemorrhoids in varying doses and compare the results to untreated individuals at risk of developing hemorrhoids to determine which doses are effective for prophylaxis against hemorrhoids.

The naproxen may be in a controlled-release form, as discussed above. The naproxen may be administered orally, topically, rectally, or using other routes of administration known in the art.

In certain embodiments, the method comprises administering to the individual naproxen in a dose of from about 0.25 mg/kg/day to about 20 mg/kg/day. In other embodiments, the method comprises administering to the individual naproxen in a dose of from about 0.5 mg/kg/day to about 5 mg/kg/day. In some embodiments, the method comprises administering to the individual naproxen in an amount between about 50 mg and about 1000 mg per day. In other embodiment, the method comprises administering to the individual naproxen in an amount between about 100 mg and bout 880 mg per day. In other embodiments, the method comprises administering to the individual naproxen in an amount between about 220 mg and about 660 mg per day.

In certain embodiments, the method comprises administering naproxen to the individual in a dose of from about 220 mg to about 660 mg. In some embodiments, the method comprises administering naproxen to the individual in a 220 mg dose. In other embodiments, the method comprises administering naproxen to the individual in a 440 mg dose. In yet other embodiments, the method comprises administering naproxen to the individual in a 660 mg dose.

In some embodiments, naproxen is administered daily. In other embodiments, naproxen is administered more than once a day. In certain embodiments, naproxen is administered twice a day. In other embodiments, naproxen is administered three times a day. In some embodiment, the method comprises administering naproxen to the individual at a twelve-hour interval. In other embodiments, the method comprises administering naproxen to the individual at a six-hour interval. In yet another embodiment, naproxen is administered in a dose of 220 mg every day.

In some embodiments, naproxen is administered at a two-day interval. In some embodiments, naproxen is administered at a three-day interval. In certain embodiments, naproxen is administered in a dose of 440 mg at a three-day interval.

In certain embodiments, administering naproxen to the individual is prophylactic against the development of a hemorrhoid symptom in the individual. In some embodiments, administering naproxen to the individual is prophylactic against development of a hemorrhoid symptom in the individual that is not pain or inflammation. In some embodiments, administering naproxen to the individual is prophylactic against development of anal itching. In other embodiments, administering naproxen to the individual is prophylactic against development of bleeding from the rectal area. In other embodiments, administering naproxen to the individual is prophylactic against development of pus discharge from the rectal area. In other embodiments, administering naproxen to the individual is prophylactic against development of a foul odor associated with hemorrhoids. In other embodiment, administering naproxen to the individual is prophylactic against development of lumps near the anus.

To further illustrate these embodiments, the following examples are provided. These examples are not intended to limit the scope of the claimed invention, which should be determined solely on the basis of the attached claims.

### EXAMPLE 1

### Treatment of Individuals Suffering from Hemorrhoids

Five individuals suffering from hemorrhoids were selected for treatment with naproxen. The group included men and women between the ages of 38 and 68, and weighing between 122 and 210 pounds. The initial cause of the hemorrhoids, as reported by the individuals, varied from individual to individual and included changes in diet, a colonoscopy, childbirth, straining during a bowel movement, and sitting for extended periods of time.

The individuals with hemorrhoids were treated with 440 mg of naproxen sodium, three times daily at six-hour intervals. Treatment was continued until symptoms of hemorrhoids abated. All treated individuals reported a complete lack of hemorrhoid symptoms after treatment, except one individual, who reported a significant improvement in symptoms.

### EXAMPLE 2

### Reversing the Progression of Hemorrhoids

After completing treatment in Example 1, three of the individuals at risk for hemorrhoids were treated at the onset of hemorrhoid symptoms to determine whether naproxen could halt or reverse the progression of the hemorrhoid symptoms.

At the first noticeable symptom of hemorrhoids, the individuals were treated with 660 mg of naproxen sodium. Thereafter, the individuals received 440 mg of naproxen sodium, three times daily at six-hour intervals. Treatment was continued until the emerging symptoms of hemorrhoids abated.

All of the individuals treated at the onset of hemorrhoid symptoms reported that the symptoms did not worsen, and that all symptoms of hemorrhoids had completely disappeared within 12-24 hours of the commencement of treatment.

### EXAMPLE 3

### Prophylaxis Against Hemorrhoids

Individuals at risk for hemorrhoids were treated prophylactically with naproxen sodium before the onset of hemorrhoid symptoms to determine whether naproxen is effective in prophylaxis against hemorrhoids.

Two individuals from Example 1 did not participate in Example 2, but instead took 440 mg naproxen sodium at three-day intervals.

Over the course of one and a half years, individuals in Example 2 experienced emerging symptoms of hemorrhoids, and needed to take naproxen to halt and reverse the progression of these symptoms. On the other hand, neither of the individuals taking 440 mg naproxen every third day experienced any symptoms of hemorrhoids.

Additionally, one individual from Example 2 was instructed to begin taking 220 mg naproxen sodium every day to determine whether a lower dose, every day dosing schedule, would be equally effective in preventing hemorrhoids. Since beginning the new treatment regimen, the individual has not experienced any emerging hemorrhoid symptoms. These data are consistent with a prophylactic effect of naproxen.

It will be obvious to those having skill in the art that many changes may be made to the details of the above-described embodiments without departing from the underlying principles of the invention. The scope of the present invention should, therefore, be determined only by the following claims.

1. A method of treating an individual suffering from hemorrhoids, comprising administering to the individual a therapeutically effective amount of naproxen, wherein administering the naproxen to the individual leads to abatement of a hemorrhoid symptom in the individual.

2. The method of claim 1, wherein naproxen is administered to the individual in a dose of from about 1 mg/kg/day to about 40 mg/kg/day.

3. The method of claim 1, wherein naproxen is administered to the individual in a dose of from about 4 mg/kg/day to about 10 mg/kg/day.

4. The method of claim 1, wherein the naproxen is administered to the individual at a twelve-hour interval.

5. The method of claim 1, wherein the naproxen is administered to the individual at a six-hour interval.

6. The method of claim 1, wherein the naproxen is administered to the individual once, twice, or three times per day.

7. The method of claim 1, wherein 220 mg naproxen is administered to the individual twice per day.

8. The method of claim 1, wherein 250 mg naproxen is administered to the individual twice per day.

9. The method of claim 1, wherein 275 mg naproxen is administered to the individual twice per day.

10. The method of claim 1, wherein 375 mg naproxen is administered to the individual twice per day.

11. The method of claim 1, wherein 440 mg naproxen is administered to the individual twice per day.

12. The method of claim 1, wherein 500 mg naproxen is administered to the individual twice per day.

13. The method of claim 1, wherein 550 mg naproxen is administered to the individual twice per day.

14. The method of claim 1, wherein 440 mg naproxen is administered to the individual twice per day, at a twelve-hour interval.

15. The method of claim 1, wherein 440 mg naproxen is administered to the individual three times per day, at a six-hour interval.

16. The method of claim 1, wherein the naproxen is administered orally.

17. The method of claim 1, wherein the naproxen is administered topically.

18. The method of claim 1, wherein the naproxen is administered rectally.

19. A method of treating an individual suffering from hemorrhoids, comprising administering to the individual a therapeutically effective amount of a controlled-release form of naproxen, wherein administering the controlled-release form of naproxen to the individual leads to abatement of a hemorrhoid symptom in the individual.

20. (canceled)

21. (canceled)

22. A method of prophylaxis against hemorrhoids in an individual at risk of developing hemorrhoids, comprising administering to the individual a therapeutically effective amount of naproxen, wherein administering the naproxen to the individual is prophylactic against the development of a hemorrhoid symptom in the individual.

23-33. (canceled)
